(19) United States
(12) Patent Application Publication
(10) Pub. No.: US 2007/0270821 A1
(43) Pub. Date: Nov. 22, 2007

(54) Vertebral stabilizer

(75) Inventors: Hai H. Trieu, Cordova, TN (US); Michael C. Sherman, Memphis, TN (US); Jason Eckhardt, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/413,448

(22) Filed: Apr. 28, 2006

(51) Int. Cl. A61F 2/30

(52) U.S. Cl. 606/61

(57) ABSTRACT

A bio-compatible stabilization system includes one or more inserters and a connector for traversing a space between one or more bony structures. The stabilization system is designed to reduce or eliminate stress shielding effects while functioning as a tension band. The elastic properties of the connector can be selected and set on a per-patient basis to allow variance in range of motion.

### BACKGROUND

Severe back pain and nerve damage may be caused by injured, degraded, or diseased spinal joints and particularly, spinal discs. Current methods of treating these damaged spinal discs may include vertebral fusion, nucleus replacements, or motion preservation disc prostheses. Disc deterioration and other spinal deterioration may cause spinal stenosis, a narrowing of the spinal canal and/or the intervertebral foramen, that causes pinching of the spinal cord and associated nerves. Current methods of treating spinal stenosis include laminectomy or facet resection. Alternative and potentially less invasive options are needed to provide spinal pain relief.

### SUMMARY

In one aspect, this disclosure is directed to a connector having more than one elongated members. The elongated members, although connected to two common ends, have different lengths and different elastic properties.

In another aspect, a single connector composed of multiple annular sections is disclosed. The single connector is constructed such that any of the annular sections may be engaged by an inserter designed to position the single connector adjacent an anchor securable to a bony structure.

In a further aspect, a spinal stabilization kit is disclosed as having a single, bio-compatible connector designed to be anchored to a first anchor and a second anchor by a first pedicle screw and a second pedicle screw, respectively. The single connector has a first plurality of tension sections and a second plurality of tension sections wherein the first plurality of tension sections has a diameter different than that of the second plurality of tension sections.

Another aspect of the disclosure is directed to a spinal stabilization system that includes a connector of substantially constant diameter extending between a first end and a second end. The connector has either a non-sequential or sequential connection of elastic and inelastic components.

In accordance with another aspect, the disclosure is directed to a system for stabilizing a spinal motion segment. The system includes a first anchor and a second anchor, as well as a tension member connected to the first anchor and the second anchor and sized to span a distance between at least two vertebral bodies. The tension member is designed to allow limited displacement of the first anchor and the second anchor from one another. The system further has a fiber member connected to the first anchor and the second anchor and sized to span the distance between the at least to vertebral bodies. The fiber member provides an increasing resistance to limited displacement of the first anchor and the second anchor from one another as the fiber member is extended from a relatively relaxed state to a relatively taut state.

In another aspect, a surgical method is disclosed as including securing a first pedicle screw to a first vertebral body and anchoring one end of a connector to the first pedicle screw. The connector has a first tension member and a second, different from the first, tension member. The surgical method further comprises securing a second pedicle screw to a second vertebral body spaced from the first vertebral body and anchoring another end of connecting body to the second pedicle screw with more tension applied to the first tension member and than in the second tension member.

These and other aspects, forms, objects, features, and benefits of the present invention will become apparent from the following detailed drawings and descriptions.

### BRIEF DESCRIPTION OF THE DRAWINGS

FIG. 1 is a pictorial representation of a vertebral column with a vertebral stabilizing system according to one embodiment of the present disclosure.

FIG. 2A is a partial perspective view of a connector in a relatively relaxed condition according to one embodiment of the present disclosure.

FIG. 2B is a partial perspective view of the connector of FIG. 2A with a tensile force applied thereon according to one embodiment of the present disclosure.

FIG. 3A is a partial perspective view of a connector in a relatively relaxed condition according to another embodiment of the present disclosure.

FIG. 3B is a partial perspective view of the connector of FIG. 3A with a tensile force applied thereon according to another embodiment of the present disclosure.

FIG. 4A is a partial perspective view of a connector in a relatively relaxed condition according to a further embodiment of the present disclosure.

FIG. 4B is a partial perspective view of the connector of FIG. 4A with a tensile force applied thereon according to a further embodiment of the present disclosure.

FIG. 5A is a partial perspective view of a connector in a relatively relaxed condition according to yet another embodiment of the present disclosure.

FIG. 5B is a partial perspective view of the connector of FIG. 5A with a tensile force applied thereon according to another embodiment of the present disclosure.

FIG. 6A is a partial elevational view of a connector according to yet a further embodiment of the present disclosure.

FIG. 6B is a cross-sectional view of the connector of FIG. 6A taken along line 6B-6B.

FIG. 7A is a partial elevational view of a connector according to one embodiment of the present disclosure.

FIG. 7B is a cross-sectional view of the connector of FIG. 7A taken along line 7B-7B.

FIG. 8A is a partial cross-sectional view of a connector according to another embodiment of the present disclosure.

FIG. 8B is a partial cross-sectional view of the connector of FIG. 8A with a tensile force applied thereon according to another embodiment of the present disclosure.

FIG. 9A is a partial cross-sectional view of a connector according to an embodiment of the present disclosure.

FIG. 9B is a partial cross-sectional view of the connector of FIG. 9A with a tensile force applied thereon according to one embodiment of the present disclosure.

FIG. 10A is a partial cross-sectional view of a connector according to another embodiment of the present disclosure.

FIG. 10B is a partial cross-sectional view of the connector of FIG. 10A with a tensile force applied thereon according to another embodiment of the present disclosure.

### DETAILED DESCRIPTION

The present disclosure relates generally to the field of orthopedic surgery, and more particularly to systems and methods for stabilizing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

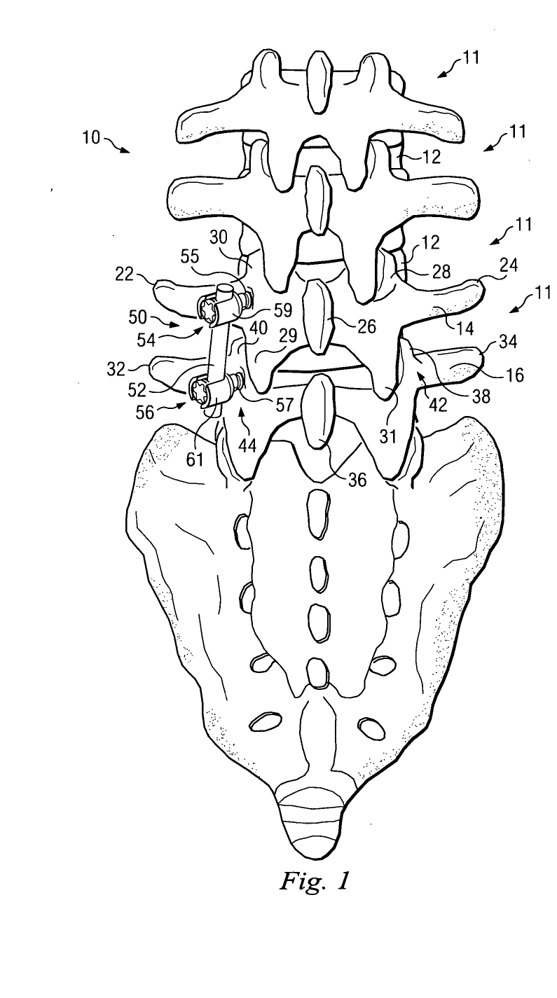

Referring to FIG. 1, the numeral 10 refers to a spinal column having a series of vertebral joints 11, each including an intervertebral disc 12. One of the vertebral joints 11 will be described further with reference to adjacent vertebrae 14, 16. The vertebra 14 includes transverse processes 22, 24; a spinous process 26; superior articular processes 28, 30; and inferior articular processes 29, 31. Similarly, the vertebra 16 includes transverse processes 32, 34; a spinous process 36; superior articular processes 38, 40; and inferior articular processes (not labeled). Although the illustration of FIG. 1 generally depicts the vertebral joint 11 as a lumbar vertebral joint, it is understood that the devices, systems, and methods of this disclosure may also be applied to all regions of the vertebral column, including the cervical and thoracic regions. Furthermore, the devices, systems, and methods of this disclosure may be used in non-spinal orthopedic applications.

A facet joint 42 is formed, in part, by the adjacent articular processes 31, 38. Likewise, another facet joint 44 is formed, in part, by the adjacent articular processes 29, 40. Facet joints also may be referred to as zygapophyseal joints. A healthy facet joint includes a facet capsule extending between the adjacent articular processes. The facet capsule comprises cartilage and synovial fluid to permit the articulating surfaces of the articular processes to remain lubricated and glide over one another. The type of motion permitted by the facet joints is dependent on the region of the vertebral column. For example, in a healthy lumbar region, the facet joints limit rotational motion but permit greater freedom for flexion, extension, and lateral bending motions. By contrast, in a healthy cervical region of the vertebral column, the facet joints permit rotational motion as well as flexion, extension, and lateral bending motions. As the facet joint deteriorates, the facet capsule may become compressed and worn, losing its ability to provide a smooth, lubricated interface between the articular surfaces of the articular processes. This may cause pain and limit motion at the affected joint. Facet joint deterioration may also cause inflammation and enlargement of the facet joint which may, in turn, contribute to spinal stenosis. Removal of an afflicted articular process may result in abnormal motions and loading on the remaining components of the joint. The embodiments described below may be used to stabilize a deteriorated facet joint while still allowing some level of natural motion.

Injury, disease, and deterioration of the intervertebral disc 12 may also cause pain and limit motion. In a healthy intervertebral joint, the intervertebral disc permits rotation, lateral bending, flexion, and extension motions. As the intervertebral joint deteriorates, the intervertebral disc may become compressed, displaced, or herniated, resulting in excess pressure in other areas of the spine, particularly the posterior bony elements of the afflicted vertebrae. This deterioration may lead to spinal stenosis. In one application, the embodiments described below may restore more natural spacing to the posterior bony elements of the vertebrae, decompress an intervertebral disc, and/or may relieve spinal stenosis. Referring still to FIG. 1, in one embodiment, a vertebral stabilizing system 50 may be used to provide support to the vertebrae 14, 16, at least partially decompress the disc 12 and the facet joint 44, and/or relieve stenosis.

Connected at each end to vertebral fasteners 54, 56, a flexible connector 52 may provide compressive support and load distribution, providing relief to the intervertebral disc 12. In addition, the flexible connector 52 may dampen the forces on the intervertebral disc 12 and facet joint 44 during motion such as flexion. Because the flexible connector 52 is securely connected to the vertebral fasteners 54, 56, the flexible connector 52 also provides relief in tension. Accordingly, during bending or in extension, the flexible connector 52 may assist in providing a flexible dampening force to limit the chance of overcompression or overextension when muscles are weak. In addition, the flexible connector 52 allows at least some torsional movement of the vertebra 14 relative to the vertebra 16. In one exemplary embodiment, the fasteners 54, 56 include a pedicle screw 55, 57 that together with anchors 59, 61 secure the flexible connector 52 in place. Such an exemplary fastener is described in U.S. Patent App. Pub. No. 2005/0277922, the disclosure of which is incorporated herein by reference.

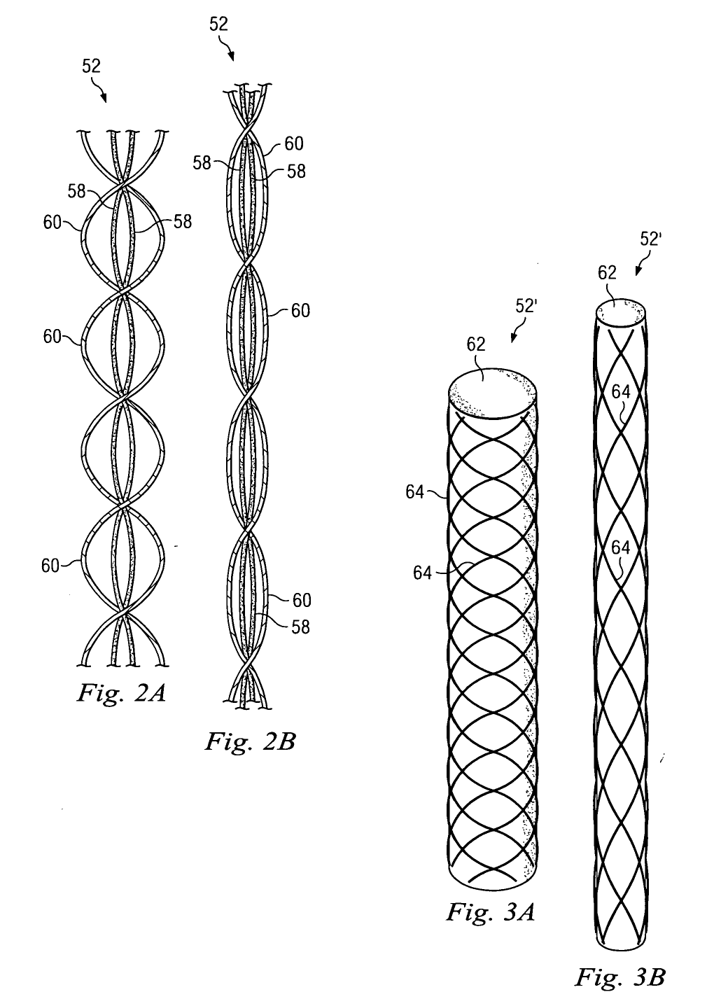

FIGS. 2A-2B show one exemplary embodiment of the flexible connector 52 in greater detail. In this embodiment, the flexible connector 52 includes a braid of elastic and inelastic fibers 58, 60, respectively. It is understood that “elastic” and “inelastic” are relative terms and the materials used for the inelastic fibers may have an elasticity albeit reduced relative to the elasticity of the elastic materials. It is also contemplated that a desired elasticity response for the connector 52 may be achieved by utilizing various types of elastic and inelastic materials in a single implementation. That is, the invention is not limited to a connector having a single type of elastic fiber and a single type of inelastic fiber. In addition to variations in fiber type, it is also understood that the fibers may have varying thicknesses, shapes, geometries, and the like. It is also understood that suitable materials and/or configurations other than or in addition to fibers may be used, including, but not limited to wires, coils, threads, filaments, twines, and combinations thereof.

In the embodiment illustrated in FIG. 2A, a braid of fibers is at a relatively relaxed state. In this regard, there is play or slack in the inelastic fibers 60. When the elastic fibers 58 are stretched in response to displacement of the fasteners from one another, the slack in the inelastic fibers 60 will be taken, as illustrated in FIG. 2B. As the distance between the fasteners 54, 56 increases, the amount of slack in the inelastic fibers 60 decreases. When substantially all of the slack is taken-up, the inelasticity of the taut inelastic fibers will resist further displacement of fasteners 54, 56 from one another. This resistance to further displacement is additive with the resistance to stretching present in the elastic fibers 58. As a result, the exemplary connector operates according to a non-linear stress-strain curve. In other words, the connector is constructed such that the resistance to an applied tensile force exists in a non-linear pattern to limit excessive motion and the onset of instability.

Referring now to FIGS. 3A-3B, a flexible connector according to another exemplary embodiment of the invention is shown. Flexible connector 52′ has a generally cylindrical elastic core 62 with inelastic fibers 64 wrapped in a sleeve therearound. The inelastic fibers may be bonded or otherwise sealed to the outer surface of the elastic core or, alternatively, held in a relatively loose engagement with core 62.

As shown in FIG. 3B, when a tensile force is applied to the connector, the elastic properties of the elastic core 62 allow the elastic core to stretch. Contrastingly, the inelastic fibers wrapped around the elastic core will not stretch, i.e., fiber length remains fixed, but the fibers are drawn together and, while doing so, compress against the outer surface of the elastic core. Accordingly, the compression of the inelastic fibers against the elastic core provides an increased resistance to displacement of the fasteners from one another.

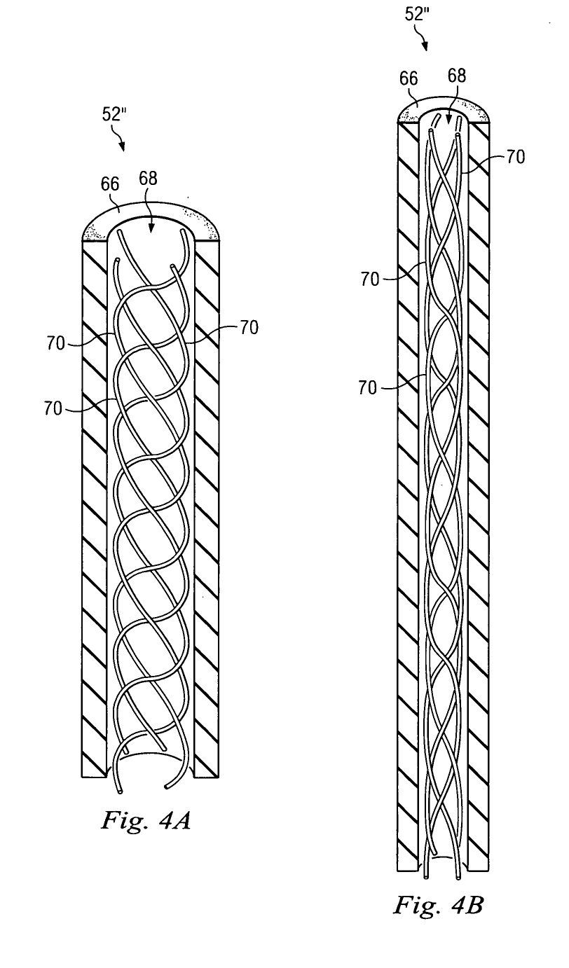

Another exemplary embodiment for the connector is shown in FIGS. 4A-4B. The connector 52″ has an elastic sleeve 66 defining a passageway 68 therethrough. The passageway provides a housing for inelastic fibers 70 that extend lengthwise through the elastic sleeve. The inelastic fibers extend through the passageway in a slacked manner and, in this regard, have a length that is greater than that of the elastic sleeve. Similar to the embodiments described with respect to FIGS. 2A-3B, the slack in the inelastic fibers provides an increased resistance to tensile forces applied on the elastic sleeve as the elastic sleeve is stretched and the fibers are drawn taut, as illustrated in FIG. 4B. It is not required that the inelastic fibers be of similar type, composition, length, diameter, geometry, and the like. Moreover, the elastic shell may be formed of homogenous or inhomogeneous material(s).

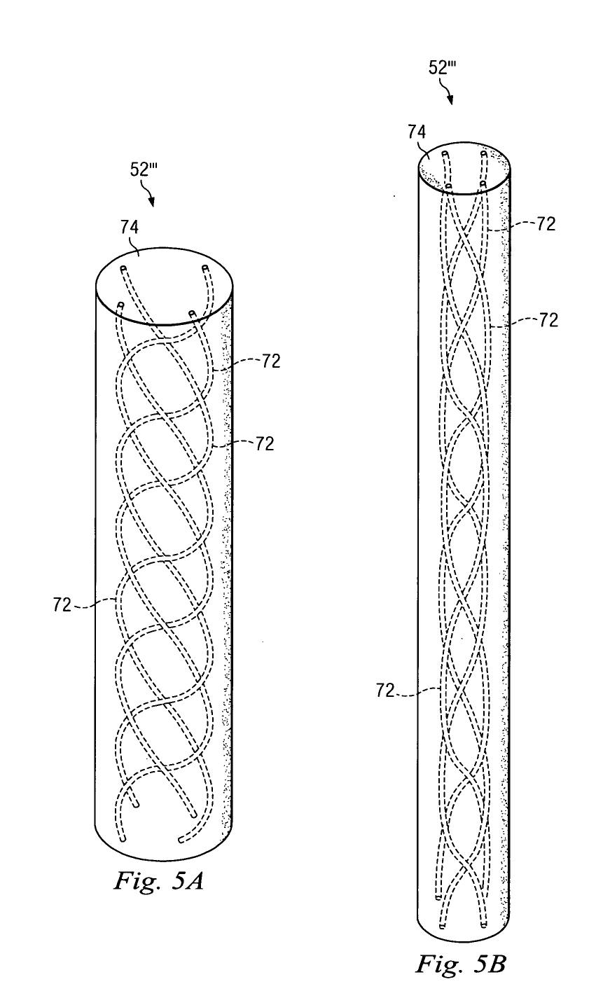

Another connector according to the present invention is shown in FIGS. 5A-5B. In this alternate connector 52′″, inelastic fibers 72 are embedded within an elastic core 74. The inelastic fibers are preferably embedded in a slacked manner during fabrication of the elastic core 74. As such, when a tensile force is applied to the connector and the elastic core is stretched, the inelastic fibers will resist the applied force as the fibers are drawn taut. Thus, as shown in FIG. 5B, when the elastic core is stretched, the core compresses and the previously slacked fibers will be drawn into a more taut condition.

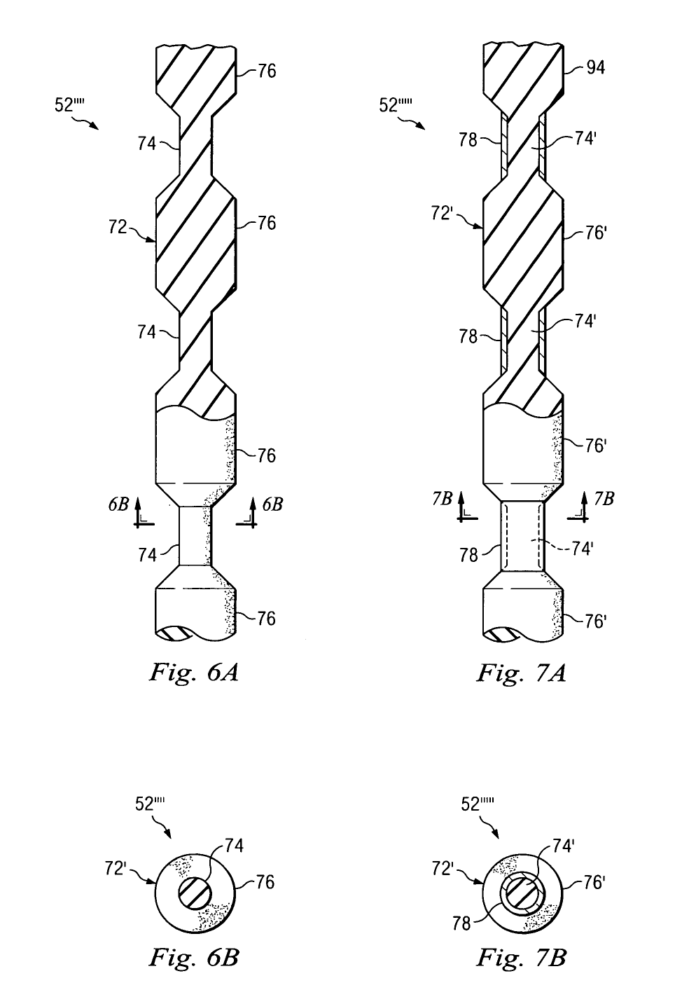

In the embodiments heretofore described, the connector has included an inelastic component and an elastic component. In the alternate embodiment shown in FIGS. 6A-6B, the connector is of single piece construction. Connector 52″″ has an elongated body 72 that is defined by multiple smaller-diameter annular sections 74 and larger-diameter sections 76. In the illustrated embodiment, when the connector is in a relatively relaxed state, the radial differences between the annular sections is well-pronounced. However, as a tensile force is applied to the connector, the body and, as a result, the annular sections are stretched. The smaller-diameter sections 74 are designed to stretch more than the larger-diameter sections 76. As such, the elasticity of the larger-diameter sections 76 is more resistive to the tensile force applied on the connector body.

In one exemplary embodiment, connector 52″″ is made of a homogenous material; however, the invention is not so limited. It is understood that all or part of each annular section may have a homogenous or heterogeneous make-up. It is understood that the connector 52″″ may have a solid-cored body, as shown in cross-section in FIG. 6B, or a hollowed or semi-hollowed (e.g., honeycombed) cored body. It is also contemplated that portions of the connector body may be solid whereas other portions may be hollow or semi-hollow.

Referring now to FIGS. 7A-7B, another exemplary embodiment of a connector according to the present disclosure is shown. In this alternate embodiment, connector 52′″″ is similar in construction to the connector of FIGS. 6A-6B with a connector body 72′ and multiple annular sections 74′ and 76′. However, in this alternate embodiment, sleeves 78 are placed circumferentially around one or more of the smaller-diameter annular sections 74′. The annular rings 78 are preferably of relatively rigid construction and therefore provide improved engagement with the screws (55 and 57, FIG. 1).

In one exemplary embodiment, connector 52′″″ is made of a homogenous material; however, the invention is not so limited. It is understood that all or part of each annular section may have a homogenous or heterogeneous make-up. It is understood that the connector 52′″″ may have a solid-cored body, as shown in cross-section in FIG. 7B, or a hollowed or semi-hollowed (e.g., honeycombed) cored body. It is also contemplated that portions of the connector body may be solid whereas other portions may be hollow or semi-hollow.

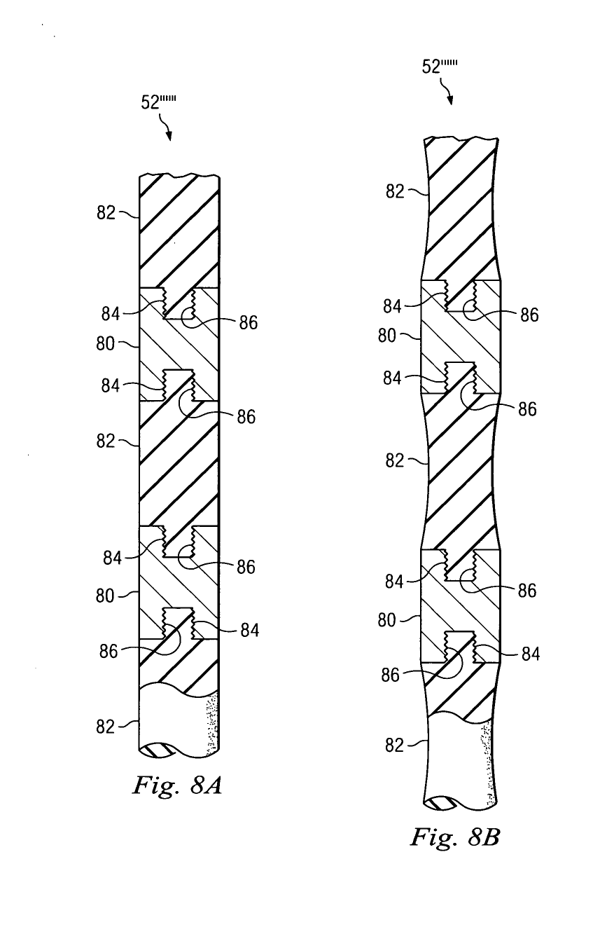

In yet another alternate embodiment, a constant diameter, multi-component connector 52″″″ includes a series of elastic and inelastic sections engaged or otherwise connected to one another. As illustrated in FIGS. 8A-8B, the inelastic sections 80 and the elastic sections 82 are threadingly connected to one another. In the illustrated embodiment, each elastic section 82 has a threaded shaft 84 at opposite ends thereof. Each inelastic section 80 has mating channels 86, each of which is threaded in such a manner to snuggly mate to a threaded shaft of an adjacent inelastic section.

It is contemplated that the elastic sections and inelastic sections may be constructed to each have a threaded shaft on one end and a receiving channel on an opposite end. In other words, each section may have a male end and a female end. With this construction, a surgeon is given increased flexibility in putting together the sequential components of the connector. That is, unlike the connector shown in FIGS. 8A-8B, with each section having a male end and a female end, a surgeon can connect two or more elastic sections together or two or more inelastic sections together in a sequential or non-sequential (randomized) manner.

It is understood that a threaded engagement is but one contemplated means for connecting the inelastic and elastic sections to one another. It is also contemplated that quick-connect connections, snap-fit connections, and other interlocking connections may be used. Additionally, bonding and other adhesive-based connections may be used in place of or in addition to mechanically locking connections.

Similar to the embodiments heretofore described, connector 52″″″ is constructed to operate according to a non-linear stress-stain curve. In this regard, the inelastic sections 80 remain fixed in length while the elastic sections 82 stretch in response to a tensile force applied on the connector. As shown in FIG. 8B, this variability in elasticity characteristics along the length of the connector causes the elastic sections 82 to stretch whereas length of the inelastic sections 80 remain unchanged.

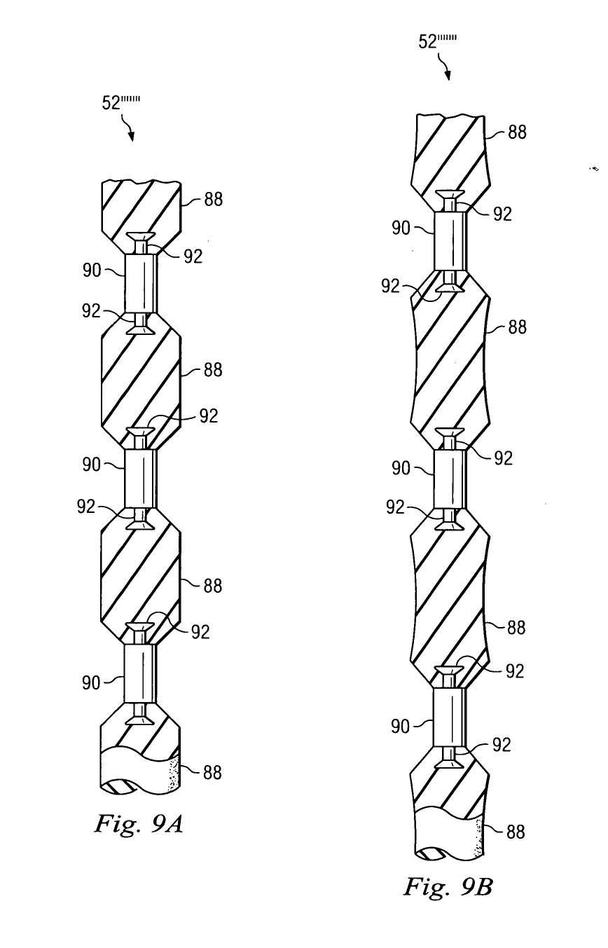

Referring now to FIGS. 9A-9B, connector 52′″″″ has multiple elastic 88 and inelastic sections 90 connected to one another via relatively inflexible flanges 92. As shown, the inelastic sections 90 have flanges that extend into the elastic sections 88. The flanges provide an increased surface area for bonding adjacent sections to one another. With a secure bond between adjacent sections, when a tensile force is applied to the connector, the elastic sections 88 will elongate whereas the inelastic sections 90 remain fixed in their length, as is shown in FIG. 9B.

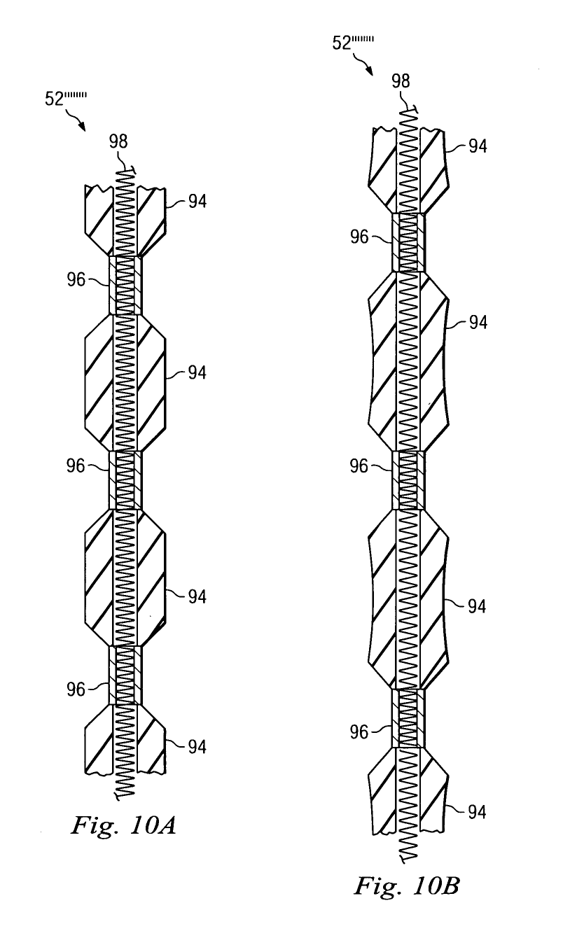

The connector 52″″″″ shown in FIGS. 10A-10B illustrates another embodiment of the present disclosure. Connector 52″″″″ has alternating elastic 94 and inelastic sections 96 linearly arranged along its length. It is contemplated that elastic and inelastic sections are bonded together or may be formed as a single unitary body. Additionally, the connector 52″″″″ has a coiled cable 98, such as a spring, extending lengthwise through the connector body. Cable 98 is preferably bonded or otherwise secured to the inelastic sections 96. In this regard, as shown in FIG. 10B, when a tensile force is placed on the connector, the elastic sections as well as the coiled cable will extend, but that extension will be resisted by the inelastic sections which are connected to both the coiled cable and the elastic sections.

The flexible connectors 52 described herein may be placed directly adjacent the vertebrae 14, 16, or alternatively, may be spaced from the vertebrae 14, 16. In some embodiments, placement of the flexible connector 52 directly adjacent the vertebrae 14, 16 may impart specific characteristics to the flexible connector 52. In some examples, the flexible connector 52 may be spaced from the vertebrae 14, 16. Accordingly even when the vertebral column is in flexion, causing the spine to bend forward, the first and second vertebral fasteners 54, 56 maintain a line of sight position, so that the flexible connector 52 extends only along a single axis, without bending. In other examples, after placement, the flexible connector 52 may contact portions of the vertebrae 14, 16 during the flexion process. For example, during flexion, the vertebrae 14, 16 may move so that the first and second vertebral fasteners 54, 56 do not have a line of sight position. Accordingly, the flexible connector 52 may be forced to bend around a protruding portion of the vertebrae. This may impart additional characteristics to the flexible connector 52. For example, because the flexible connector 52 would effectively contact the spinal column at three locations (its two ends 62, 64 and somewhere between the two ends), its resistance to extension might be increased.

In the exemplary embodiments described, the flexible connector 52 is the only component extending from one vertebral fastener 54, 56 to the other. This may be referred to as a single flexible connector. This single flexible connector may be contrasted with conventional systems that employ more than one connector extending between attachment points, such as systems with one component connected at the attachment points and another component extending between attachment points. Because it employs a single flexible connector 52, the vertebral stabilizing system 50 disclosed herein may be easier and quicker to install, may be less complex, and may be more reliable than prior devices.

It should be noted however, that a spinal column may employ the flexible connector 50 to extend across a first vertebral space, with a second flexible connector extending across a second vertebral space. Accordingly, more than one vertebral stabilizing system 50 may be used in a spinal column. In some instances where more than one stabilizing system is use, the first and second vertebral spaces may be adjacent. In alternative embodiments, a vertebral stabilizing system 50 may have a single flexible connector with a length allowing it to extend across more than one intervertebral space, with or without connecting to an intermediate vertebra.

In certain anatomies, the vertebral stabilizing system 50 may be used alone to provide decompression or compression to a single targeted facet joint or to relieve pressure on a particular side of the intervertebral disc, such as a herniation area. However, in some instances, a second vertebral stabilizing system may be installed on the opposite lateral side of the vertebrae 14, 16, across from the vertebral stabilizing system 50. Use of first and second vertebral stabilizing systems may provide more balanced support and equalized stabilization. The second vertebral stabilizing system may be substantially similar to system 50 and therefore will not be described in detail.

The vertebral stabilizing system 50, as installed, may flexibly restrict over-compression of the vertebrae 14, 16, thereby relieving pressure on the intervertebral disc 12 and the facet joint 44. In addition, the vertebral stabilizing system 50 may flexibly restrict axial over-extension of the intervertebral disc 12 and the facet joint 44. By controlling both compression and extension, the vertebral stabilizing system 50 may reduce wear and further degeneration. The flexible connector 52 may also dampen the forces on the intervertebral disc 12 and facet joint 44 during motion such as flexion and extension. Because the flexible connector 52 may be positioned relatively close to the natural axis of flexion, the vertebral stabilizing system 50 may be less likely to induce kyphosis as compared to systems that rely upon inter-spinous process devices to provide compressive and tensile support. Additionally, the system 50 may be installed minimally invasively with less dissection than the inter-spinous process devices of the prior art. Furthermore, an inter-pedicular system can be used on each lateral side of the vertebrae 14, 16, and may provide greater and more balanced stabilization than single inter-spinous process devices.

It should be noted that in some embodiments, the flexible connector 52 may be configured so that orientation in one direction provides one set of stabilizing properties to the vertebrae, while orienting the flexible connector 52 in the other direction would provide a second set of stabilizing properties. In such an embodiment, the body 58 of the flexible member may be asymmetrically shaped.

It should be noted that the flexible connector 52 can be made of elastic or semi-elastic materials in parts or in its entirety. On the other hand, the connector 52 can be made of a composite of elastic/semi-elastic and inelastic or rigid materials. Exemplary elastic materials include polyurethane, silicone, silicone-polyurethane, polyolefin rubbers, hydrogels, and the like. The elastic materials can be resorbable, semi-resorbable, or non-resorbable. Exemplary inelastic materials include polymers, such as polyetheretherketone (PEEK), polyetherketoneketone (PEKK), and polylactic acid materials (PLA and PLDLA), metals, such as titanium, NITINOL, and stainless steel, and/or ceramics, such as calcium phosphate and alumina. Further, the various connector components can be solid, hollow, semi-hollow, braided, woven, mesh, porous, or combinations thereof. The connector can also be reinforced or semi-reinforced.

Although disclosed as being used at the posterior areas of the spine, the flexible connector may also be used in the anterior region of the spine to support the anterior column. In such a use, the flexible connector may be oriented adjacent to and connect to the anterior column, and may span a vertebral disc space.

The foregoing embodiments of the stabilization system may be provided individually or in a kit providing a variety of sizes of components as well as a variety of strengths for the connector. It is also contemplated that the connector's characteristics may be color coded or otherwise indicated on the connector itself to expedite identification of a desired connector.

The invention is also embodied in a surgical method for spinal or other bone stabilization. In accordance with this method, a surgeon performs a conventional interbody fusion/nucleus replacement/disc replacement followed by placement of pedicles/bone screws or other inserters into appropriate vertebral or other bony structures. The surgeon may then anchor one end of a connector into a first vertebral or other bony structure. If necessary or otherwise desired, tension is applied to the connector spanning the space between bony structures. Preferably, tension is applied in a limited manner so that inelastic components of the connector are imposing little or no resistance on the applied tension. The un-anchored end of the connector is then anchored to a second vertebral or other bony structure spaced from the first vertebral or other bony structure. Any excess connector extending past the inserters is preferably cut and removed.

As referenced above, various embodiments of the connector described herein include disjointed sections that can be threadingly engaged or otherwise connected to each other on a per patient basis. Thus, the above surgical method contemplates a surgeon connecting a desired number of elastic and inelastic segments to each other until a desired length, elasticity, and the like is achieved. Moreover, as shown above, a surgeon can construct such a connector on-the-fly quickly and with relative ease.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and can be varied within the scope of the disclosure. Further, the embodiments of the present disclosure may be adapted to work singly or in combination over multiple spinal levels and vertebral motion segments. Also, though the embodiments have been described with respect to the spine and, more particularly, to vertebral motion segments, the present disclosure has similar application to other motion segments and parts of the body. In the claims, means-plus-function clauses are intended to cover the elements described herein as performing the recited function and not only structural equivalents, but also equivalent elements.

We claim:

1. An implant for stabilizing bony structures, the implant comprising:
a first end and a second end;
a first elongated member extending between the first end and the second end and traversing a space between at least two bony structures, the first elongated member having a first elasticity and a first length; and
a second elongated member extending between the first end and the second end and traversing the space between the at least two bony structures, the second elongated member having a second elasticity different from the first elasticity and a second length different from the first length.

2. The implant of claim 1 wherein the second length is greater than the first length.

3. The implant of claim 1 wherein the second elongated member includes a plurality of substantially inelastic fibers.

4. The implant of claim 3 wherein the plurality of substantially inelastic fibers are constructed in either a braided or woven arrangement.

5. The implant of claim 1 wherein the first elongated member includes an elastic sleeve and wherein the second elongated member is disposed within a bore defined by the elastic sleeve.

6. The implant of claim 5 wherein the second elongated member is bonded to an inner surface of the elastic sleeve.

7. The implant of claim 1 wherein the first elongated member extends circumferentially around the second elongated member.

8. The implant of claim 1 wherein the second elongated member extends circumferentially around the first elongated member.

9. The implant of claim 1 wherein the first elongated member includes a substantially solid rod of elastic material and wherein the second elongated member is embedded within the substantially solid rod.

10. The implant of claim 1 configured to operate according to a non-linear stress-strain elasticity curve.

11. The implant of claim 1 wherein the first elongated member is formed of bio-compatible material(s).

12. The implant of claim 1 wherein the second elongated member is formed of a metal or a ceramic.

13. The implant of claim 1 further comprising a pair of anchors, a respective anchor disposed at the first end and the second end, each anchor configured to receive an inserter for operably securing the first and the second elongated members relative to the bony structures.

14. The implant of claim 1 wherein the bony structures are vertebral bodies.

15. A implant system comprising:
a single connector having a first annular section and a second annular section different in diameter and linearly displaced from the first annular section; and
an inserter designed to engage the single connector to position the single connector adjacent an anchor securable to a bony structure;
wherein the single connector is constructed such that either the first annular section or the second annular section may be engaged by the inserter.

16. The implant system of claim 15 wherein the single connector is formed of elastic material.

17. The implant system of claim 16 wherein the first annular section has a first elasticity and the second annular section has a second elasticity different from the first elasticity.

18. The implant system of claim 16 further comprising an inelastic sleeve disposed circumferentially around the first annular section.

19. The implant system of claim 16 wherein the single connector is comprised of a homogeneous material.

20. The implant system of claim 15 wherein the single connector is securable to the anchor absent a stabilizing tether extending therethrough.

21. The implant system of claim 15 wherein the first annular section and second annular section are bonded to one another.

22. The implant system of claim 15 wherein the first annular section and the second annular section are threadingly connected to one another.

23. The implant system of claim 15 further comprising a first inserter and a second inserter different from the first inserter, and wherein the single connector in use extends between the first inserter and the second inserter with multiple first annular sections and multiple second annular sections present along a length of extension.

24. The implant system of claim 15 wherein the single connector has a length sufficient to span a space between adjacent bony structures.

25. The implant system of claim 15 wherein the single connector has a length sufficient to span a distance between non-adjacent bony structures.

26. The implant system of claim 15 wherein the first annular section comprises elastic material and the second annular section comprises inelastic material.

27. A spinal stabilization kit comprising:
a first bone anchor and a second bone anchor; and
a single, bio-compatible connector designed to be anchored to the first bone anchor and the second bone anchor, the single connector having a first plurality of tension sections and a second plurality of tension sections, the first plurality of tension sections having a diameter different than that of the second plurality of tension sections.

28. The kit of claim 27 wherein the single connector is formed of a homogeneous elastic material.

29. The kit of claim 28 wherein the connector further comprises a plurality of sleeves, each sleeve circumferentially extending around a respective tension section of the first plurality annular sections.

30. The kit of claim 28 wherein the single connector has a substantially solid core.

31. The kit of claim 30 wherein the first plurality of tension sections is formed of substantially rigid core material and the second plurality of tension sections is formed of substantially elastic core material.

32. The kit of claim 27 wherein the single connector is configured to allow limited displacement of the first anchor from the second anchor when engaged thereto.

33. The kit of claim 27 wherein the single connector is formed by threadingly engaging tension sections to one another.

34. A spinal stabilization system comprising:
a first end and a second end;
a connector of substantially constant diameter extending between the first end and the second end, the connector having either a sequential or non-sequential connection of elastic and inelastic components.

35. The system of claim 34 wherein adjacent elastic and inelastic components are bonded to one another.

36. The system of claim 35 wherein an adjacent elastic and inelastic components are threadingly engaged to one another.

37. A system for stabilizing a spinal motion segment, the system comprising:
a first anchor and a second anchor;
a tension member connected to the first anchor and the second anchor and sized to span a distance between at least two vertebral bodies, the tension member designed to allow limited displacement of the first anchor and the second anchor from one another; and
a fiber member connected to the first anchor and the second anchor and sized to span the distance between the at least to vertebral bodies, the fiber member providing an increasing resistance to limited displacement of the first anchor and the second anchor from one another as the fiber member is extended from a relatively relaxed state to a relatively taut state.

38. The system of claim 37 further comprising a first screw engageable with the tension member, the fiber member, and the first anchor and a second screw engageable with the tension member, the fiber member, and the second anchor.

39. The system of claim 37 wherein the tension member includes an inner passage having the fiber member extending therethrough.

40. The system of claim 37 wherein the tension member includes a solid core embedded with the fiber member.

41. The system of claim 37 wherein the fiber member is formed as an inelastic core for the tension member.

42. The system of claim 41 wherein the inelastic core is bonded to an inner surface of the tension member.

43. The system of claim 37 wherein the fiber member has a length greater than that of the tension member.

44. The system of claim 37 wherein the tension member and the fiber member extend between the first anchor and the second anchor in a braided arrangement.

45. The system of claim 37 wherein the fiber member is constructed as a sleeve extending circumferentially around the tension member.

46. The system of claim 37 wherein the tension member and the fiber member are connected to the first and the second anchors absent a tether member extending between the first and the second anchors.

47. A surgical method comprising the steps of:
implanting a first bone anchor to a first vertebral body;
securing one end of a connector to the first bone anchor, the connector having a first tension member and a second, different from the first, tension member;
implanting a second bone anchor to a second vertebral body spaced from the first vertebral body; and
securing another end of the connector to the second bone anchor with tension applied to the first tension member and slack in the second tension member.

48. The surgical method of claim 47 further comprising the step of extending the connector before securing the another end to apply tension thereto.

49. The surgical method of claim 48 wherein the step of extending is carried out such that the connector spans a distance between two adjacent vertebral bodies.

50. The surgical method of claim 48 wherein the step of extending is carried out such that the connector spans a distance between two non-adjacent vertebral bodies.
